(19) United States
(12) Patent Application Publication
(10) Pub. No.: US 2009/0217569 A1
(43) Pub. Date: Sep. 3, 2009

(54) Method for Lipid production

(75) Inventors: Ossi Pastinen, Kantvik (FI); Simo Laakso, Turku (FI); Sanna Hokkanen, Espoo (FI); Miia Mujunen, Espoo (FI)

Correspondence Address: DODDS & ASSOCIATES, 1707 N STREET NW, WASHINGTON, DC, 20036, US

(73) Assignee: Helsinki University of Technology, Espoo (FI)

(21) Appl. No.: 12/291,946

(22) Filed: Nov. 14, 2008

Related U.S. Application Data
(60) Provisional application No. 61/003,056, filed on Nov. 14, 2007.

(51) Int. Cl. C10L 1/18; C12P 7/64

(52) U.S. Cl. 44/308; 435/134

(57) ABSTRACT

The invention relates to a method for forming a lipid or a lipid mixture from an organic source material comprising cellulose, hemicellulose, starch, non-starch polysaccharide, any mixture thereof or degradation products thereof. According to the method, the source material is treated one or more times with water, an aqueous solution of acid or alkali, and the precipitate and the filtrate are separated. The precipitate obtained from the treatments mentioned above can be subjected to mechanical or thermo-mechanical grinding, or the precipitate can be treated with a strong acid, or the precipitate can be acidified and ground mechanically or thermo-mechanically. After the treatments, the precipitate and the filtrate are separated. A lipid-producing microorganism is contacted with the source material or the obtained filtrate(s) in a culture medium, whereby the microorganism cells begin to produce lipid, and the lipids are recovered.

The invention relates to a method of producing a lipid or a lipid mixture from organic raw materials according to the preamble of Claim 1. The invention also relates to the use of the lipid or the lipid mixture produced by the method as a biofuel according to Claim 18, as well as to a biofuel according to Claim 20. The invention also relates to a method of purifying municipal sewage according to Claim 22.

### BACKGROUND

It is commonly known that the use of traffic fuels manufactured from fossil raw materials is extremely large-scale and that the consumption is continuing to grow. Thus, the adequacy, the environmental effects, and the aspect of sustainable development concerning fossil energy resources have quite rightly emerged as essential global challenges. In this frame of reference, the renewable alternative raw materials of traffic fuels have emerged as an object of increasing interest.

One step towards fuel production based on renewable natural resources is to attempt, at least partly, to replace the fossil raw materials with organic materials. Even with this approach, one can envision problems that are fairly difficult to solve. In proportion to the present consumption of fossil raw materials, the amount of organic raw material required is extremely large, even if only a portion of the fossil raw materials are to be replaced. In several connections, it has already been observed that a unilateral and large-scale consumption of organic natural resources or the reclaiming of areas under cultivation for this purpose may result in consequences to the biodiversity of nature and to the balance of the primary production of foodstuffs, which will be difficult to solve. The conversion of the organic material into a form that can be used for the production of traffic fuel in an energy-effective manner also presents a technological challenge.

A particularly favourable raw material source for traffic fuels would be organic fat, particularly triacyl glycerol as its energy content is considerably higher than that of corresponding carbohydrates or alcohols, for example. Furthermore, it is the best-known and can be converted into components of traffic fuel, such as diesel fuel, biodiesel or renewable diesel, through relatively effective chemical processes. However, the scarcity of the reserves of natural fatty raw materials presents a limiting factor. Based on present fatty resources, no more than a marginal industrial production of biofuel is feasible, at the most. Thus, increasing the fat reserves requires quite a considerable increase in the cultivation of fat plants. Such a large change in the production sector of cultivation towards fat plants, in turn, has a strong influence on the balance of the food economy on the global market. This need, still at a speculative level, is already manifesting itself as a heavy rise in the prices of food and forage raw materials.

The total amount of naturally renewable organic masses is quite large; calculated as an amount of carbon, considerably larger than the annual use of fossil carbon as traffic fuel. However, the major part of these renewable masses, about 60%, consists of compounds, which still contain a substantial amount of oxygen and whose fuel value is thus quite low.

On the basis of prior known technology, it is known that the utilization of the relatively low energy content of carbohydrates into higher-energy compounds containing more reduced hydrocarbon chains is already in theory, and particularly as known chemical applications, such as the gasification technique, an extremely energy-intensive process (R. Agrawal, N. R. Singh, F. H. Ribeiro and W. N. Delgass 2007. “Sustainable fuel for the transportation sector”, PNAS 104: 4828-4833, and WO 2006/117317), wherein the total efficiency between the feed and the yield remains low. A corresponding basic problem is also associated with biotechnological processes according to the known technology, which are used to convert the hexose sugars contained in carbohydrates into higher-energy compounds. An example of this is the production of alcohols, particularly ethanol, which is described, among others, in the publications US 2002/0185447, U.S. Pat. No. 5,637,502, and WO 03/038067.

The patent publication US 2004/0231661 also describes the treatment of a material containing lignocellulose by means of water and acid extractions and by means of hydrolysis, so that xylose and glucose are formed, which can be used in the preparation of ethanol. The patent publication U.S. Pat. No. 5,221,357 describes the treatment of a material containing hemicellulose and cellulose by means of acid hydrolysis, and the treatment of the solid phase mechanically and by means of acid hydrolysis to produce monosaccharides, such as pentose and hexose sugars, which could be used in the preparation of ethanol. The patent publication U.S. Pat. No. 4,752,579 describes the treatment of the husks of corn seeds to separate monosaccharides by means of acid and/or alkali, and enzymatic hydrolysis.

Some publications describe the production of lipids by means of microbial fermentation from different organic materials. The patent publication U.S. Pat. No. 4,368,056 suggests the use of carbohydrates, which exist in low contents in industrial waste materials, in butanol fermentation, and the use of microbial glycerides, which are generated in the fermentation, in the production of biodiesel. The publication Dai et al. (Dai, C. Tao, J., Xie, F., Dai, Y. and Zhao, M. Biodiesel generation from oleagenous yeast Rhodotorula glutinis with xylose assimiating capacity. Afr. J. Biotechnol. Vol 6 (18), pp. 2130-2134, 19 Sep. 2007) describes the extraction of carbohydrates that are present in the straw of plant material, such as corn stems, tree leaves and rice, by means of grinding and an acid treatment, and the use of the filtrates and washing waters, obtained from the treatments, as raw material for the production of lipid by the Rhodotorula yeast. The lipid obtained from the microbe was used in the production of biodiesel. Angerbauer et al. (Angerbauser, C., Sierbenhofer, M. Mittelbach, M. and Guebtz, G. M. Conversion of sludge into lipids by Lipomyces starkeyi for biodiesel production. Bioresource Technology 99 (2008) 3051-3056), in turn, describes the treatment of waste water sludge by alkali and acid, as well as its utilization in lipid production by the Lipomyces yeast, into a raw material for biodiesel.

The problems encountered with the methods described above include the fact that the yields of sugars that can be utilized by the micro-organisms remain low, or the methods apply to the use of biomaterials as source material that are of minor importance to the needs of large-scale production. It is obvious that the quantitative goals set for the manufacture of biofuel cannot be achieved by means of the described methods and the source materials used therein.

Thus, there is still a great need for new technological solutions, particularly solutions that could be used, more comprehensively than before, to convert the renewable organic carbohydrate reserve of the globe into compounds having a higher energy content, irrespective of its chemical or structural composition. It would be of particular importance to solve how carbohydrates existing in various organic materials, could be effectively converted into compounds having a higher energy content, such as fats, which would be more adaptable to traffic fuel use or to the raw material of this use.

### SUMMARY

The purpose of the present invention is to provide a new solution to the problem of how to convert organic biomaterial into compounds having a higher energy content.

In particular, the purpose of the present invention is to provide a solution to the problem of how to convert the carbohydrate components obtainable from organic biomaterial into a lipid suitable for the production of biodiesel.

To be more precise, the method according to the present invention is characterized by what is stated in the characterizing parts of Claim 1.

The use according to the invention, in turn, is characterized by what is stated in Claim 18, and the biofuel by what is stated in Claim 20.

The method for purifying municipal sewage according to the invention is characterized by what is stated in Claim 22.

The present invention is based on the observation that, when handling biomass in different ways for the recovery of cellulose and hemicellulose fractions, micro-organism populations appeared quicker in said fractions the further the cellulose and hemicellulose fractions were degraded. Surprisingly, it was discovered that in the carbohydrate fractions described above, micro-organisms were also growing that had the ATP citrate lyase enzyme (EC 2.3.3.8, previously EC 4.1.3.8), by means of which the organisms collected lipids, particularly triacyl glycerol, into their cells. Based on these observations, the invention developed for treating biomaterial so that the cellulose and hemicellulose contained in it are separated from the rest of the biomaterial and then hydrolyzed so that the hydrolysis products are suitable for growing lipid-collecting micro-organisms, and for the production of lipid, and to use the lipid thus formed as a raw material in the manufacture of biodiesel.

According to the description of the invention, carbohydrates suitable to be utilized by the lipid-synthesizing micro-organisms can be produced from organic raw material originating from various sources, and/or carbohydrate fractions can be produced, from which lipid can be produced by means of micro-organisms. According to the present invention, such carbohydrates can be produced particularly from biomaterials that contain hemicellulose, cellulose, starch or non-starch polysaccharides. The carbohydrates that are suitable for utilization by micro-organisms are particularly mono and oligosaccharides, which comprise both hexose and pentose sugars. The carbohydrates can also be in polymeric forms, if the lipid-producing micro-organism has been selected so that it is capable of using carbohydrate polymers.

Wood, in its various forms, comprises the biggest reserve of renewable biomass that can be recovered at present. The use of wood, particularly its mechanical or thermo-mechanical treatment or other manufacture or production of mechanical mass from wood, is large-scale and, as a process, produces a lot of carbohydrate-bearing minor flows. Very little economic added value has been discovered for these side cuts of the wood-refining industry; and in many cases, they incur costs as the environmental load caused by them must be eliminated. As a technological challenge, side flows like these are particularly problematic. The side flows are large in volume, but their carbohydrate content is typically low. As dilute water-based carbohydrate solutions, they are poorly suitable for processes that aim at utilizing the carbohydrates in solution by chemical means.

Accordingly, the purpose of the present invention is also to provide a solution to the problem of how to utilize large-scales industrial side flows containing biomaterial, which presently require costly purification procedures or remain completely unutilized by the present processes, but which still have potential as a source of energy.

In the method according to the present invention, organic source material is treated, which comprises cellulose, hemicellulose, starch, all of these, some mixture thereof or the degradation products thereof or, alternatively, starch or non-starch carbohydrate as such or linked with the cellulose or hemicellulose materials. The source material can be pre-treated mechanically, thermo-mechanically, physically, chemically, biologically or by combinations of these treatments, or it may be suitable to be used as such. When containing carbohydrates in polymeric form, it is preferably treated with water or acid or alkali, or combinations thereof. After these preliminary treatments according to the present description, the mixture is divided into a filtrate and solid matter, i.e. a precipitate (FIG. 1), and the filtrate or both fractions are recovered. It is preferable to renew the treatment of the source material that is carried out with water or acid or alkali, or their combinations, and to combine the filtrates with each other after the separation of the precipitate. The filtrate obtained in the treatment containing alkali, either as such or after the recycling described above, is preferably conveyed to a mixture, wherein an acid treatment of the source material is carried out to increase the amount of soluble monosaccharide. Any of the filtrates or precipitates generated in these treatments or the source material as such, or the combinations of the source material and the filtrate or precipitate or filtrates or precipitates are used to produce single-cell lipid after possible pre-treatments, such as neutralization, decolourization and filtering. The filtrates can also be combined, diluted or concentrated to achieve a suitable monosaccharide content and composition for the micro-organisms that produce single-cell lipid.

From each alternative treatment of the source material, a precipitate of a varying composition is generated, depending on whether the treatment is carried out with water or in the presence of acid or alkali. To increase the sugar yield, the precipitates are preferably conveyed to mechanical grinding, either as such or in the presence of water acid or alkali, from which treatment a filtrate and a precipitate are obtained again. The filtrate or the precipitate or some combination thereof is used for producing single-cell lipid, or the precipitate is treated preferably with a strong acid, when so desired. As a result of the acid treatment, a filtrate and a precipitate are generated again, from which the filtrate or the precipitate or the combination of these can be conveyed to the production of single-cell lipid. The precipitate can be treated in even higher acid concentrations by combining grinding with the treatment. The filtrate or the precipitate or the combination thereof, generated as a result of this treatment, is used for the production of single-cell lipid. The precipitate can also be removed and burned or used for the production of biofuel or a precursor thereof by other methods. Each filtrate fraction or precipitate or source material can be used as such or as various combinations for the production of single-cell lipid. The precipitates obtained from the process steps described above can be retreated by means of the earlier or later process steps presented in the present description. To increase the sugar yield, it is thus preferable to treat the precipitate with an acid or alkali that is stronger than in the previous treatment.

Further, enzyme treatments or microbial fermentations can be carried out between the different process steps.

The invention also relates to a method, wherein the used alkalis and acids are recycled again in the process.

The invention also relates to a method, wherein the single-cell biomass used in the method is recycled as the biomaterial intended by the invention, when the produced lipids have been recovered.

Economically exploitable components can be recovered from the precipitate obtained from the treatments that are carried out according to the method. The filtrates, in turn, can be used also in other microbial processes than the production of lipid.

The method according to the description provides a solution to the problem of how the hexose and pentose monosaccharides contained in the carbohydrates of the biomass can be gradually brought into smaller fractions containing a larger amount of monomeric sugar units, which fractions the lipid-synthesizing micro-organisms can utilize more effectively to produce lipid. In connection with any process step, in which an insoluble substance is separated from a soluble substance, a precipitate, and in which a filtrate is thus generated, the hexose and pentose sugars contained in the filtrate can be used for producing single-cell lipid as such or, after necessary pre-treatments, as mixtures of filtrates for the production of single-cell lipid. In addition to the filtrates, also precipitates or combinations of filtrates and precipitates can be used for the production of single-cell lipid.

A comprehensive advantage of the invention is that it can be used to apply simple processes and unit operations, which are already in industrial use, in an energy-effective and environmental manner to produce an energy-rich chemical compound, a lipid, from compounds of biological origin containing less energy, such as hexose and pentose monosaccharides or the oligomers formed by these, as well as the mixtures of these.

In the method according to the invention, it is particularly preferable that, by means of mechanical or thermo-mechanical grinding, a new surface is always exposed in the organic material, which can again be subjected to treatments with water, or acid or alkali with a different strength, and thus a solution and precipitate distribution can be provided, from which, particularly with the solution, more sugars usable in the microbial lipid production can be obtained.

When combining several separations of the solution and the precipitate, a considerably enhanced yield of hexose and pentose sugars is obtained for the mechanical or thermo-mechanical grinding, compared with single-phase hydrolyzing methods.

In the method according to the invention, by combining the filtrates obtained from the acid and alkali treatments or by combining the filtrates and the precipitates, the flows obtained from the various treatments neutralize each other. Thus, the filtrates or the precipitates or the combinations thereof are usable as such, without adjusting the acidity, or at least the need to adjust the acidity is smaller. The possibility to combine several different biomasses and biomasses from different origins with each other is also advantageous.

The exploitability of the method becomes particularly emphasized so that the sugars formed therein, which are usable in lipid production, can naturally be microbiologically used, either as such or partly, to produce also other compounds, such as alcohols.

A special advantage of the invention is that, in addition to the utilization of the side or main flows of the mechanical and thermo-mechanical treatment of wood, it is also suitable for the utilization of other biomaterials that release carbohydrates for producing lipid.

The invention further relates to a method for forming lipid or a lipid mixture from a mixture that, according to the method, was generated by recycling once again the fibre, which was generated in the thermo-mechanical treatment, in the treatments with alkali or acid.

Other organic raw materials that are difficult to utilize on a large scale, which according to the present description can be processed into hexose and pentose sugars or into oligomers formed by these and formed from these into lipid by means of micro-organisms, are recycled fibre that is obtained, for example, from the recycling of newspaper, beet pulp that is obtained from sugar beets, and the chaffs and straws of grains, such as oat; and other similar devalued part of field-cultivated plants, sawdust, refined mechanical pulp, straw and peat, particularly slightly decomposed peat. Other organic materials that so far have hardly been utilized at all are swampy or submerged biomass, biomass from the catchment areas of cellulose mills, and the biomass that goes to activated sludge plants from municipal sewage, or other organic municipal waste that goes to dumping areas or incineration. These organic materials can also be treated by alternating the various embodiments of the method so that the carbohydrates contained in these materials are rendered useful for the micro-organisms to produce single-cell biomass and lipid. For example, municipal wastewater could be treated by means of the present method, whereby there would, among others, be the advantage of harmful microbes dying in the treatment.

An essential point of the present invention is that the carbohydrate-containing biomaterial, regardless of which organic material it originates from, is treated so that monomeric hexose and/or pentose sugars or their oligomers suitable for the production of single-cell lipid are obtained from the carbohydrates. It is preferred to carry out the treatment by a combination of two or more treatments.

Using the method according to the invention, suited to the dimensions of the needs for large-scale production of biofuel, source materials can be simultaneously produced from several different biomasses, which source materials are usable in the production of the cellmass of lipid-producing micro-organisms and in the production of lipid. The components suitable for the lipid production of microorganisms can be produced irrespective of the composition, availability and structure of the biomass. A considerable advantage of the present invention is also that, by the method according to the invention, usable sugars can be produced with a high yield and the consumption of chemicals needed for the adjustment of acidity can be reduced.

Compared to the existing state of the prior art, the invention described herein provides a breakthrough technique, which combines the conversion of biomaterials that contain both cellulose and hemicellulose into usable hexose and pentose sugars. By the same embodiments, the invention also enables the utilization of the monosaccharide units of the starch and the non-starch polysaccharides contained in these biomaterials into usable sugars for the production of single-cell lipids. The invention is particularly implementable so that it can be applied on an industrial scale to materials, which originate from renewable natural resources or their devalued side flows, which are generated by the industry or communities. The method according to the invention can be used to treat material containing cellulose and hemicellulose in a controlled manner, so that precursors of single-cell lipids are formed therefrom, which can be used by safe microorganisms in the production of single-cell lipids.

In the following, the invention is described more closely by means of the appended drawings and a detailed description.

### BRIEF DESCRIPTION OF THE DRAWINGS

FIG. 1 shows the main steps of performance of the method according to the invention.

FIG. 2 shows the use of hexose and pentose sugars as such or in combinations for the production of cellular mass and lipid.

FIG. 3 shows the growth of yeasts and the production of lipid in a culture medium, to which a mixture has been added as a source of carbon and for the production of lipid, which mixture was generated from chaff by an alkaline treatment and by the 10% acid hydrolysis of the filtrate obtained therefrom.

FIG. 4 shows the growth of yeasts in a culture medium, to which commercially available pentose sugar was added as a source of carbon.

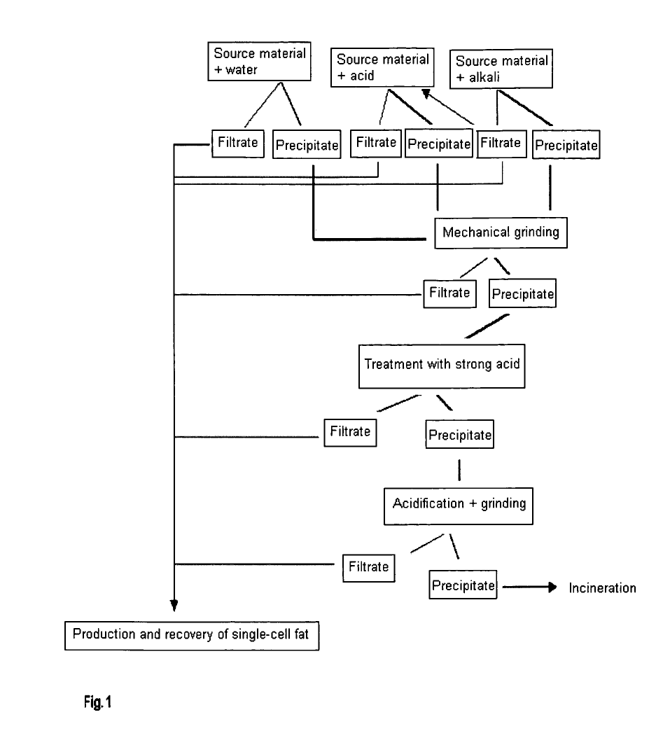

### DETAILED DESCRIPTION OF THE METHOD

“Carbohydrates” refer to organic molecules that include an aldehyde, acid or keto group and, in addition, several hydroxyl groups. The sphere of carbohydrates thus includes compounds that are described by the terms monosaccharide, oligosaccharide, polysaccharides, sugar, cellulose, hemicellulose, starch and non-starch carbohydrate.

“Cellulose” is a long-chain polysaccharide, whose primary structure consists of a polymer formed by the β-1-4 bonds of glucose.

“Starch” is a long-chain polysaccharide that mainly consists of α-1-4 and α-1-6 glucose units.

“Usable sugar” herein refers to sugars, using which the microorganisms are able to multiply, and from which the lipid- and alcohol-producing microorganisms are capable of producing lipid or alcohols.

“Hemicellulose” refers to a group of compounds consisting of several different hexose and pentose sugars, such as galactose, mannose, glucose, xylose and arabinose.

“Monosaccharide” is a monomer unit of carbohydrates, (C—H₂O)n, which typically consists of 3-9 carbon atoms and which has stereochemical differences in one or more carbon atoms. These include hexoses, such as glucose, galactose, mannose, fructose, which have 6 carbon atoms, and pentoses, such as xylose, ribose and arabinose, which have 5 carbon atoms.

“Oligosaccharide” refers to a carbohydrate, which has been formed from two or more monosaccharides by O-glycosidic bonds.

“Pentose sugar” refers to a monosaccharide containing five carbon atoms.

“Hexose sugar” refers to a monosaccharide containing six carbon atoms.

“Hydrolysis” refers to carbon-carbon, carbon-oxygen, carbon-nitrogen, or carbon-sulphur bonds breaking under the influence of either water, acid or alkali, irrespective of whether the water participates in the reaction. In an enzymatic hydrolysis, the corresponding reactions are catalyzed by enzymes. Hydrolysis is, for example, a reaction, where the O-glycosidic bond between the monosaccharides of the carbohydrates or the peptide bond between the amino acids of proteins breaks.

“Treatment with water-acid or alkali” in this connection means that an organic material either as such, or a product derived from it is extracted, treated mechanically or thermomechanically, or combinations of these treatments are carried out in the presence of water, acid or alkali. Acid refers to a chemical substance, molecule or ion, which is capable of donating a hydrogen ion (a proton), and alkali refers to a substance, molecule or ion, which is capable of accepting the hydrogen ion (the proton), according to the Brφnsted-Lowry acid alkali theory. Acid also refers to the so called Lewis acid, which is capable of accepting an electron pair, and the Lewis alkali refers to the so called Lewis alkali, which is capable of donating a base pair. According to the definitions, the activity of the substances as acids or alkalis is not limited to aqueous solutions. In the present description, the terms “acid” and “alkali”, according to the definitions, also refer to acid and alkali catalysts. In the present description, acid also refers to any acid phase, wherein it can function as acid, such as in the form of gas, solid matter or liquid; for example, as an aqueous solution. Correspondingly, alkali refers to any alkali phase, wherein it can function as an alkali, such as in the form of gas, solid matter or liquid; for example, as an aqueous solution.

“Organic source material” in the present description refers to any organic matter that is produced by a living organism. The organic source material in the present description is also called biomaterial.

In particular, the organic source material refers to an organic material that comprises polysaccharide. “Polysaccharide” refers to a carbohydrate polymer formed from monosaccharides, which can also contain compounds other than monosaccharide. Polysaccharides are, for example, cellulose, hemicellulose and starch. Polysaccharides also include, among others, alginate, glucane, inulin and arabic gum. Out of other polysaccharides, mannane should also be mentioned. The source material can comprise polysaccharides as such or as a mixture, or it may comprise their degradation products.

“Non-starch polysaccharide” refers to a carbohydrate, whose molecular structure lacks the α-1-4 bonds typical of starch, or they are scarce. Non-starch polysaccharides are, for example, glucane, alginate, inulin and arabic gum. The non-starch polysaccharides also include hemicellulose and cellulose. Other non-starch polysaccharides are, for example, the carbohydrate polymers that occur in algae.

“Cultivated plant” refers to a plant, which is planted or seeded for beneficial purposes in the soil that is prepared for it.

The term “lipid” refers to a fatty substance, whose molecule generally contains, as a part, an aliphatic hydrocarbon chain, which dissolves in organic solvents but is poorly soluble in water.

In the present invention, the lipids that are formed in the micro-organisms are mainly tri-, di- or mono-acylglycerols, or sterol esters, but other lipids, such as phospholipids, free fatty acids, sterols, polyprenols, sfingolipids, glycolipids and diphosphatidyl glycerol can also be formed in the cells.

The present invention can be used in the manufacture of biodiesel or renewable diesel.

According to the EU directive 2003/30/EY, “biodiesel” refers to a methyl-ester produced from vegetable or animal oil, of diesel quality to be used as biofuel.

Renewable diesel refers to a hydrogen-treated lipid of animal, vegetable or microbial origin, whereby the microbial lipid can originate from a bacterium, a yeast, a mould, an alga or another microorganism.

The source material of the method according to the invention can be cellulose, hemicellulose and biomass, preferably wood pulp, possibly containing binders, which has been generated by mechanical or thermo-mechanical methods or other physical methods, or chemically, enzymatically or microbiologically or by combinations of these methods. Without modifying the method, plant materials that contain starch, such as potato, its parts, the seeds of cultivable crops, maize and rise, respectively, sugar beet as well as, in addition, sugar beat pulp including the non-starch polysaccharides contained in the same, can also be the source material of the method according to the invention. Parts of plants that contain non-starch polysaccharides, such as β-glucan, are also suitable as source material. The method is also suited for the use of carbohydrates, such as alginate, which originate from single-cell organisms, as source material. The composition of the source materials described above can also include varying amounts of protein and lipid, which can also act as source materials for the growth of lipid-synthesizing microorganisms and for the lipid production.

The source material of the method according to the invention can also be, for example, recycled fibre obtained from newspaper recycling, sugar beet pulp and the chaffs of grains, such as oat, sawdust, refined mechanical pulp, peat and straw. Other source materials useful in the method according to the invention are, for example, microbial mass, such as single-cell biomass, swampy or submerged biomass, including algae and micro-algae, biomass from the catchment areas of cellulose mills, and the biomass that goes to activated sludge plants from municipal sewage, or other municipal waste that contains a biological component and that is presently used in incineration, composting or in some other method, which results in the comprehensive release of the carbon contained in the waste as carbon dioxide.

The method according to a preferred embodiment of the invention comprises at least one step, wherein the filtrate or the combination of filtrates, the precipitate or the combination of precipitates, which is obtained from the organic material according to the method, the organic material as such or a combination of any of these, is conveyed to the mixture, where the lipid production takes place. The alternative embodiment of the method can be selected on the basis of what kind of a monosaccharide composition is preferred in the filtrate or the combinations of filtrates, or in the precipitates, the combinations of precipitates, or in the combinations of filtrates or precipitates for growing the microorganism and producing the lipid. Thus, the filtrates or precipitates are selected from a group that is generated by treating the biomaterial preferably with a substance that is selected from the group comprising:
i) water,
ii) acid, and
iii) alkali, and
   by thereafter separating the fibre-containing precipitate and the fibre-free filtrate.

Optionally, the precipitate is one or more times again subjected to treatment(s) of any of the items (i), (ii) or (iii), and the precipitate obtained is preferably subjected to mechanical or thermo-mechanical grinding, and the precipitate and the filtrate are separated.

Depending on which biomaterial is used and which monosaccharides are desired, the biomaterial is treated with water, acid or alkali, preferably acid or alkali; typically, by means of an aqueous solutions of acid or alkali. When needed, the treatment can also be carried out several times. The same biomaterial can also be treated sequentially with several different solutions, and compounds that enhance the separation and hydrolyzation of the carbohydrates can be added to the water.

The source materials that are listed in the following list in Group I can be treated with water in the first step or, if the treatment result is to be enhanced, with a mixture of water and acid.

The biomaterials of Group II are treated in the first step, preferably with acid.

The biomaterials of Group III are treated in the first step, preferably with alkali. When the precipitate and the filtrate have been recovered, the filtrate can be re-treated with acid.

### Group I

Biomaterial originating from mechanical, thermomechanical, enzymatic or microbiological treatment of wood or from combinations of these treatments, or from cultivated plants.

### Group II

Recycled fibre, beet pulp, chaffs, straw, bran, grain granule, whole cultivated plant, cultivated plant, TMP pulp, MDF pulp or a source material containing starch or non-starch polysaccharides

### Group III

Sawdust, refined mechanical pulp, chaffs, straw and woody plant parts, TMP pulp, MDF pulp, beet pulp, cultivated plant, which may contain varying amounts of starch.

The treatments can be enhanced by adding, for example, one or more enzymes into the treatment solution, preferably into a treatment solution that is made using water. Enzyme treatments or microbial fermentations can also be added between the various process steps.

In the following step, the lipid-producing micro-organism is contacted with any of the filtrates or precipitates or their combinations in a culture medium, and the microorganism cells are allowed to produce lipid, and the lipids are recovered.

The fibre-containing precipitate obtained from the above-described biomaterial treatment steps is also preferably treated using a method, wherein the fibre-containing precipitate is mechanically ground and the fibre-containing precipitate and the fibre-free filtrate are separated.

In addition, the fibre-containing precipitate obtained from the mechanical grinding can be treated with a strong acid, and the fibre-containing precipitate and the fibre-free filtrate can be separated. After the acid treatment, the precipitate can also again be recycled back to the mechanical grinding or, as stated above, the precipitate can be used in lipid production using microbes.

In addition, the biomaterial can be acidified and ground mechanically or thermo-mechanically, and the fibre-containing precipitate and the fibre-free filtrate can be separated.

The filtrate or precipitate or their combination, obtained from any of the above described treatments can be added to the culture medium of the lipid-producing microorganisms.

Typically, the total amount of sugars in the filtrates is 0.5-10% by weight. Of these, sugars usable for the production of biomass and lipids typically comprise at least 0.5% by weight, preferably at least 3% by weight, more preferably 4-5% by weight. For example, by grinding and re-extracting the biomaterial, more sugars can be detached from the biomaterial and, in this way, the amount of sugars can be increased to a more advantageous level. However, the amount of sugars in the filtrate is preferably less than 30% by weight, more preferably less than 20% by weight.

The microorganisms that are capable of producing lipid can be grown so that they first produce biomass and then lipid, or simultaneously both biomass and lipid.

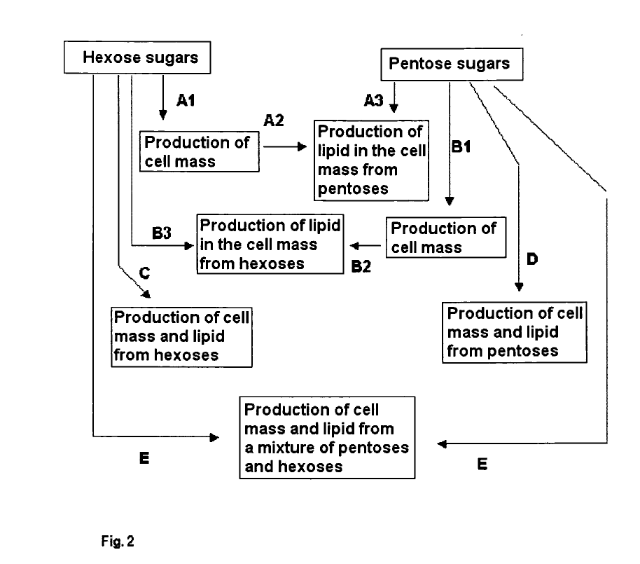

Depending on the origin of the biomaterial and the method of treatment it is subjected to (treatments with water, acid, alkali), hexose monosaccharides, pentose monosaccharides or both in various ratios can be obtained, as shown in FIG. 2. From some biomaterials, mainly hexose sugars can be obtained, whereas from others, mainly pentose sugars. With a suitable selection of the microorganism capable of producing lipid, a filtrate or precipitate or a combination thereof that mainly contains hexose sugars can be used for the production of cellular mass and, after that, a filtrate or precipitate or a combination thereof that mainly contains pentose sugars can be used for the production of lipid into the cellular mass. Alternatively, lipids can be produced in the cellular mass from hexoses. The cellular mass and the lipids can be produced from hexoses. Correspondingly, by a suitable selection of the microorganism that is capable of producing lipid, a filtrate or precipitate or a combination thereof that mainly contains pentose sugars can be used for the production of cellular mass and, after that, a filtrate or precipitate or a combination thereof that mainly contains hexose sugars can be used for the production of lipid into the cellular mass. Alternatively, lipids can be produced into the cellular mass from pentoses, or the cellular mass and the lipids can be produced from pentoses. Both cellular mass and lipids can also be produced from a mixture of pentoses and hexoses.

### Treatment of Biomaterial

In the following, the treatment of biomaterial according to the preferred embodiments of the invention is described. Typically, the treatment is carried out as a combination of two or more treatments:

The source material is preferably extracted at a temperature of 90-100° C. An advantageous embodiment of the acid extraction is to use 5-10% of a mineral acid, such as sulphuric acid, or an organic acid, such as citric acid or acetic acid, and in the alkali extraction, preferably 0.5-2.0M of NaOH. The treatment time can range widely; it is preferably 1-10 hours, typically 2-8 hours, most suitably 2-4 hours. Other treatments, such as treatments with enzymes, microorganisms, oxidizing or reducing chemicals, or combinations of these treatments, can also preferably be combined with the water extraction.

According to the method, the precipitate generated in the extraction can be mechanically ground preferably at a temperature of 100-210° C., typically 150-200° C., preferably for 2-20 minutes, typically for 5-11 minutes. The pressure is preferably 68 bar. The generated mass is filtered, the filtrate is treated using the method described above in order to be suitable for the production of single-cell lipid. The precipitate can be conveyed to an acid treatment with a strong acid, which preferably is a treatment with 40-72% sulphuric acid, suitably 65-70% sulphuric acid. Normally, the treatment time is 2-8 hours, preferably 2-4 hours. The method can be implemented with any acid, by means of which a proton-catalytic hydrolysis is provided. Suitable acids are, for example, strong mineral acids, phosphoric acid, sulphuric acid, or the oxyacids of sulphur, nitrogen, chlorine, bromine and iodine.

The result of the hydrolysis is divided into a filtrate, which is treated in order to be suitable for the production of single-cell lipid, and the precipitate can be conveyed into a dilute acid solution, preferably a solution of 5-10% sulphuric acid, and grinding can be carried out at a temperature of 170-200° C. and at a pressure of 6-10 bar, for 10-20 min. The mixture is divided into a filtrate and a precipitate, of which the former is treated in order to be suitable for the production of single-cell lipid and the precipitate can be removed.

The above described embodiment of the method aims at the total use of the carbohydrate present in the source material for the production of single-cell lipid. However, starting from the way of extracting the source material, the method can also be implemented for selected parts only, for example when the fibre material of the precipitate is also to be used for other purposes. The method according to the invention is characterized by preferably comprising the steps described above in their entirety, but is not limited from carrying out part(s) of the method to an extent that deviates from the basic method, or in a unit operation order, and to the use of the monosaccharide fractions produced by these operations in the production of single-cell lipid. A process step can also be attached to the method according to the description, wherein the monosaccharide-containing fractions obtained from the source material are used for the production of single-cell biomass or ethanol, in addition to lipid.

In the following description, some methods according to the preferred embodiments of the invention are described. The same methods can also be applied to raw materials other than the ones presented in the description.

### I.

According to a preferred embodiment of the invention, wood fibres that comprise ground wood, TMP pulp, sawdust or mechanical pulp is treated according to the following constituent steps:
A. 100 g of wood fibre is extracted in a litre of water at 90-100° C. for 2-4 hours, preferably 2 hours. The precipitate is separated from the solution by filtration, and the solution is recovered. The carbohydrate yield in the solution ranges from 2% to 5% depending on the manufacturing method of the wood fibres, typically being 4-5% for TMP fibre of a high treatment temperature (over 170° C.).
B. To increase the carbohydrate yield, the fibre fraction is hydrolyzed in a litre of 5-10% acid (preferably 5%), pH about 1 (e.g., mineral acid), at a temperature of 90-100° C. for 2-4 hours, preferably 2 hours. The remaining precipitate is separated from the solution; the solution is recovered.
C. Excess acid is preferably decanted from the precipitate, which is re-ground in a defibrator (e.g. a wing or disc refiner). It is preferred to increase the temperature of the precipitate by pre-steaming for one minute and to keep the condensate in the mixture. The temperature is raised 150-200° C., preferably 170° C., the pressure being 6-8 bar (wing refiner). The grinding time is selected according to the wood from the range of 2 to 15 minutes. The precipitate is separated from the solution by filtering and the solution is recovered.
D. Strong acid of 40-72% (sulphuric acid) is added to the precipitate fraction, it is allowed to absorb for 2-4 hours, preferably 2 hours at room temperature. Excess acid is decanted off and the precipitate is re-ground in a defibrator (e.g. a wing or disc refiner). It is preferred to increase the temperature of the precipitate by pre-steaming for one minute, without draining the condensate thereafter. The temperature is raised 150-200° C., preferably 170° C., the pressure being 6-8 bar (wing refiner). The grinding time is selected according to the wood from the range of 2 to 15 minutes. The mixture is filtered, the precipitate is separated from the solution. The solution is recovered and the precipitate is used as fuel.

The solution fraction obtained from any of the constituent steps A-D can be further processed by decolourization methods, pH adjustments, and other measures that promote the growth of microorganism, such as removing water, and the solutions thus obtained can be used in the culture medium of the microorganism, and the microorganism can be allowed to produce lipid. By combining the constituent steps A-D, 40-65% of the original wood fibre material can be converted into a soluble form from the fibre that is used as source material. The dissolved carbohydrates comprise glucose, galactose, mannose, xylose and arabinose units, their mutual portions being typical for the wood type used.

### II.

According to another preferred embodiment of the invention, 100 g of wood fibre, ground wood, recycled fibre, TMP pulp, sawdust or mechanical pulp are used, extracted in one litre of 4-8% alkali solution, preferably 1 M of NaOH at a temperature of 90-100° C. for 2-4 hours, preferably 3 hours. The precipitate is separated from the solution by filtering at room temperature, and both fractions are recovered. Depending on the treatment of the fibre of the source materials, the carbohydrate yield in the solution is within a range of 5-8%. This solution is preferably treated by any of the following constituent steps:
A. The vat liquor is re-used as such in extracting the following fibre batch in the way described above, and the solution is recovered.
B. The vat liquor is hydrolyzed with acid according to any of the steps B-D of the previous embodiment to treat the lignin and oligo- and polysaccharides that have dissolved therein.
C. The vat liquor recycled according to step A is used according to step B of embodiment I.

To increase the monosaccharide yield, the precipitate recovered in the alkali treatment is preferably treated using any of the following methods or the combinations thereof:
D. The precipitate is treated according to any of steps B, C, D of the previous embodiment I, or according to all of them. The mixture is filtered, the solution is neutralized, and both the precipitate and the solution are recovered.
E. The precipitate remaining from step A of embodiment II is re-treated with alkali, preferably under conditions, where the mixture is ground for 2-8 minutes, preferably 6 minutes, at a pressure of 4-10 bar, preferably 8 bar, the temperature being 170° C. Filtration is carried out, the solution is neutralized and recovered. By means of this extra grinding, the amount of material that is dissolved can be increased to 27% of the original amount of fibres in the source material.

The carbohydrate-containing solution generated in each constituent step A-E, comprising glucose, galactose, mannose, xylose and arabinose units, is processed into a form suitable for lipid production by microorganisms, for example, by means of decolourization, pH adjustment, or by removing water from the solution, and it is used as a culture medium for the microorganism, or as a part thereof, and the microorganism is allowed to produce lipid.

### III.

According to a third preferred embodiment of the invention, wood fibre, ground wood, TMP pulp, sawdust, mechanical pulp, recycled fibres (or a neutralized extraction prepared according to step B described above) are used, hydrolyzed with an acid that has a strength of 5-10% (preferably 5%), by adding 1 litre of said acid (e.g. a mineral acid) per 100 g of material to be hydrolyzed, at a pH of about 1, using a temperature of 90-100° C. for 2-4 hours, preferably 2 hours. The precipitate is separated from the solution by filtration. The solution contained 4-14% of carbohydrates calculated from the amount of fibres of the source material. The precipitate can be used as fibres or be further processed in order to increase the monosaccharide yield according to steps C or D or both of the first embodiment described above. The solution is separated from the precipitate, neutralized, filtered, and either used as such or in a concentrated form into the culture medium of the microorganism or a part thereof, and the microorganism is allowed to produce lipid.

### IV.

According to a fourth preferred embodiment of the invention, 100 g of wood fibres, ground wood, TMP pulp, sawdust, mechanical pulp or the precipitate remaining from steps A-C of embodiment I or step A of embodiment II are used, 5-10% of acid is added, pH about 1 (e.g. a mineral acid), it is allowed to absorb for 2-4 hours, preferably 2 hours. The excess acid is decanted off and the precipitate is re-ground in a defibrator (e.g. a wing or disc refiner). The temperature of the precipitate is preferably raised by pre-steaming for one minute, and the condensate is not allowed to flow out. The temperature is raised 150-200° C., preferably 170° C., the pressure being 6-8 bar (wing refiner). The grinding time is selected according to the wood from the range of 2 to 15 minutes. The precipitate is removed from the solution by filtration. The residual precipitate can be used as fuel or be re-ground in the way described above to increase the monosaccharide yield in the solution. The solution is neutralized, filtered and used as such or in a concentrated form, and is used in the manner described in embodiments I-III.

### V.

According to a fifth preferred embodiment of the invention, 100 g of wood fibres, ground wood, TMP pulp, sawdust, mechanical pulp or the residual precipitate according to any of embodiments I-IV is used, a strong acid of 40-72% (e.g. sulphuric acid) is added, is allowed to absorb for 2-4 hours, preferably 2 hours at room temperature. The excess acid is decanted off and the precipitate is re-ground in a defibrator (e.g. a wing or disc refiner). The temperature of the precipitate can be increased by pre-steaming for one minute, and the condensate is not allowed to flow out. The temperature is raised 150-200° C., preferably 170° C., the pressure being 68 bar (wing refiner). The grinding time is selected according to the wood from the range of 2 to 15 minutes. The carbohydrate mixture is filtered and the precipitate is separated from the solution. The portion of matter that is dissolved from the precipitate used as source material is in the range of 40-65%. The residual precipitate can be used as fuel or it can be re-ground to increase the monosaccharide yield. The solution is neutralized and filtered and treated according to any of embodiments I-IV.

Alternatively, strong sulphuric acid of 40-72% is absorbed into the precipitate at room temperature for 1-3 hours, preferably 1.5 hours. After this, the acid is diluted to 5% and boiled at normal pressure at 100° C. for 4 hours. Further treatment as above.

### Lipid Production Using Microorganisms

The present invention allows, through combination of the filtrates generated in the various constituent steps, the carbohydrate contained in the source material as well as the hexose and pentose monosaccharides to be comprehensively used for lipid and single-cell biomass by means of microbiological processes. Each recovered filtrate can, with pre-treatment, such as washing, neutralization, decolourization or other after-treatment procedures, be used as such or alternatively in combination with various aqueous fractions for the production of single-cell lipid. Because of the ways of treatment of the source material, which is part of the invention, the invention is also applicable to ethanol production.

A filtrate, i.e. an aqueous fraction, or any combination of filtrates, is added to a microorganism culture medium which has been or is inoculated with a microorganism and the microorganism is allowed to produce lipid. The lipid is recovered in the form of microorganism mass or the lipid is separated from said mass and both the lipid and the microorganism mass separated from it are recovered. Lipids can be recovered using known methods either by removing them from the cells or by disrupting the cells. The lipid can be extracted from the disrupted cells using an organic solvent. Methods of lipid recovery applicable to the invention are described, for instance, in the publication by Z. Jacob: Yeast Lipids: Extraction, Quality Analysis, and Acceptability, Critical Reviews in Biotechnology, 12(5/6); 463-91 (1992). A preferred method for recovering lipids is phase separation. The treatment of the lipid formed in the microorganism into fatty acid esters can also take place without prior homogenization of the microorganism cells and subsequent fat isolation.

A preferred embodiment of the present invention relates to a method of forming a lipid or a lipid mixture from a carbohydrate mixture generated in the processing of the organic source material, comprising hexose and pentose sugars in monomeric or oligomeric forms, according to which method the carbohydrate-containing mixture is added to an aqueous culture medium, on which a lipid-producing micro-organism is cultured, the medium is supplemented with nutrients required for the growth, inoculation of the medium with said organism is carried out, the organism is cultured and allowed to produce lipid, the cellular mass is recovered, and the lipid or the lipid mixture is separated from the cells or the fat-containing cells or their constituents are utilized as such.

The method according to the invention provides a particular flexibility for microbiological lipid production. The fraction containing both hexose and pentose sugars is a natural carbon source for many lipid-producing microorganisms. Thus, the method also has the advantage of allowing the microorganism to be selected within a wide range, for instance on the basis of lipid production capacity, yield of biomass, type of culturing or culturing conditions. The other constituents of the microorganism, besides the lipid, can be used energy-efficiently in many different ways, thus improving the overall economic performance of the process according to the invention. Preferred ways of using the lipid-free microorganism mass are hydrolysis and recycling into the culture medium of the lipid-producing microorganism or use as forage or nutritive substance. It is also possible to separate various components, such as (special) sugars, colouring agents, β-glucan, sterols, sterol esters or proteins, from the lipid-free microbial mass.

The micro-organism is selected from natural or genetically modified fat-accumulating microorganisms, preferably from yeasts, moulds, bacteria and algae, more preferably from yeasts and moulds, most preferably from yeasts. It is essential that the microorganism to be utilized is capable of producing lipid from hexose or pentose sugars or from both. The invention therefore encompasses all microorganisms in which lipid accumulation is based on the ATP:citrate lyase activity (EC 2.3.3.8) they contain.

Lipid-synthesizing yeast genera that are applicable for the invention comprise the following genera: Candida, Yarrowia, Lipomyces, Rhodotorula and Cryptococcus, which include strains that synthesize the pentose sugar xylose into lipid, such as Candida curvata (D) (Evans, C. T. and Ratledge, 1983. A comparison of the oleaginous yeast, Candida curvata, grown on different carbon sources in continuous and batch culture, Lipids 18 623-629), Rhodotorula gracilis (Yoon, S., Rhim, J., Choi, S., Ryu, D. and Rhee, J. 1982. Effect of Carbon and Nitrogen Sources on Lipid Production of Rhodotorula gracilis, J. Ferment. Technol. 60, 243-246) and Rhodosporidium toruloides, Rhodotorula glutinis, Rhodotorula graminis, Lipomyces starkeyi, Lipomyces lipofer, Candida lipolytica, Cryptococcus, Cryptococcus albidus, Trichosporon cutaneum and Trichosporon pullulans (Fall, R., Phelps, P. and Spindler, D. 1984, Bioconversion of Xylan to Triglycerides by Oil-Rich Yeasts. Appl. Environ. Mircobiol. 47, 1130-1134) as well as a strain that synthesizes the pentose sugar arabinose into lipid, Lipomyces starkeyi (Naganuma, T., Uzuka, Y. and Tanaka K. 1985. Physiological Factors Affecting Total Cell Number and Lipid Content of the Yeast, Lipomyces starkeyi, J. Gen. Appl. Microbiol. 31, 29-37).

Correspondingly, the fat-accumulating mould genera that are applicable to the invention comprise, among others:
Aspergillus
Chaetomium
Clodosporidium
Cunninghamella
Emericella
Fusarium
Mortierella
Mucor
Penicillium
Pythium
Rhizopus
Trichoderma

Correspondingly, the fat-accumulating bacterial genera that are applicable to the invention comprise, among others:
Acinetobacter
Actinobacter
Anabaena
Arthrobacter
Bacillus
Clostridium
Flexibacterium
Micrococcus
Mycobacterium
Nocardia
Nostoc
Oscillatoria
Pseudomonas
Rhodococcus
Rhodomicrobium
Rhodopseudomonas
Shewanella
Streptomyces
Vibrio

Correspondingly, the fat-accumulating micro-algae genera that are applicable to the invention comprise, among others:
Botryococcus
Brachiomonas
Chlamydomonas
Chlorella
Crypthecodinium
Dunaliella
Euglena
Nannochloris
Nannochloropsis
Navicula
Nitzschia
Schizochytrium
Sceletonema
Scenedesmus
Tetraselmis
Thraustochytrium
Ulkenia

According to a preferred embodiment of the invention, microorganisms that synthesize fatty acid-containing lipid into their cells in an amount, which preferably is 12-65% by weight of the dry weight of the cells, are used for the lipid synthesis.

According to a particularly preferred embodiment of the invention, the lipid-free biomass formed in the invention, treated in a way suited for the microorganism, is used as nutrients in the culture medium. In addition to these components, the culture medium can be supplemented with components preferable for the microorganism employed. In order to produce lipid, the microorganism generally requires, among others, a carbon source, which it in the present invention acquires from the source material, a nitrogen source, such as an inorganic ammonium salt (e.g. ammonium sulphate) or an organic nitrogen source (e.g. amino nitrogen, yeast extract, or hydrolyzed cellular mass), and a micronutrient source, such as a phosphate, sulphate, chloride, vitamin or cation source (e.g. a Mg, K, Na, Ca, Fe or Cu ion source), whereby these components can be added to the medium when needed. When the method of the present invention is applied, the lipid concentration of the cells is preferably 40% by weight, most preferably 65% by weight.

### Manufacture of Biofuel

The fatty acid ester contained in the microorganism-derived lipid can be treated to be suitable as biodiesel fuel by any known method. One preferred approach is to carry out a transesterification with short-chained alcohols, preferably methanol, to obtain the alcohol ester of the fatty acid.

The impure side flow generated in the manufacture of biodiesel or renewable diesel taking place with transesterification, which side flow contains alcoholic compounds, such as glycerol or non-esterified fatty acid salt, and is difficult to exploit in a energy-efficient way, can be re-used for the production of single-cell lipid, which lipid is usable as such or recyclable into other glycerolipid-containing materials of biological origin.

### ADVANTAGES OF THE INVENTION

The advantages of the invention include the fact that the equipment needed for the method is simple and the associated technology is known as far as manufacture and operation are concerned. The method according to the invention is not limited to any production scale, but can be easily scaled up or down according to the carbohydrate content and amount of the source material to be treated. Carrying out the method to produce lipid does not require energy-consuming heating, pressurized unit operations or other chemical catalysts in addition to acid, alkaline or enzyme catalysts. The method only requires the use of chemicals that can be incorporated in the internal cycle of the method according to the invention, or the processing of such biomaterials. Nor does the method require cost-increasing water-removal from the usable sugar solutions, since dilute carbohydrate solutions are suitable, as such, for use in or as the microorganism culture medium. The overall economic performance of the method is improved by the fact that the lipid-free biomass generated in it has, in addition to the internal cycle, many different uses, such as the production of individual organic constituents, as forage or as the raw material of the forage, or as a supplementary culture medium for the production of microorganisms. The method is also suited for the production of single-cell biomass and ethanol.

Advantages of the stagewise treatment of biomass according to the present invention are the more comprehensive hydrolysis of organic material and, thus, the better usability of the organic material for the production of single-cell lipid compared to the present technology. Further, the filtrates or precipitates obtained from the acid and alkali treatments neutralize each other and reduce the use of chemicals required in a neutralization.

The solutions of the prior art describe methods containing microbiological steps for ethanol production, wherein carbohydrate-containing materials are used as raw materials, as such or converted into monosaccharides. Patent application publication US 2002/0185447 and patent U.S. Pat. No. 5,637,502 also describe carbohydrate treatment methods, such as treatment with acid or alkali, these treatments being followed by alcoholic fermentation. Regarding microbiological treatment, both of the said methods are limited to ethanol production, wherein hexose sugar formed from polysaccharides is used. In patent application publication US 2003/0096385, prenyl alcohol (geranyl and farnesyl derivatives of alcohol) is produced using microorganisms. Patent application publication WO 03/038067 describes a method, wherein modification of the genome of a fungous microorganism can yield an organism capable of utilizing pentose sugars. The publication is only aimed at ethanol production.

The invention provides a new possible solution to the exploitation of the more significant aqueous side flow of thermo-mechanical wood processing particularly taking place within the pulp and paper industry, particularly as raw material for traffic fuel. The invention allows for the biological loading of the carbohydrate-containing side flow generated in connection with the manufacture of mechanical wood pulp, and hence the energy costs of wastewater treatment, to be reduced. In concrete form, the invention is a lipid production process providing an environmentally friendly solution to producing raw material for a traffic fuel, biodiesel or renewable diesel, for instance from the dilute, carbohydrate-containing aqueous fractions generated in TMP processes or corresponding mechanical treatment of wood.

The method also provides alternative solutions for the exploitation of other source materials. In accordance with the method, recycled fibre, such as printing paper, packaging material and comparable cellulose-based materials, can be used as source material.

Compared with prior art, the invention is, therefore, in compliance with the principles of sustainable development, by increasing the availability of lipid raw material and reducing the total demand for organic lipid from other sources. The invention thus enhances the availability of renewable natural resource-based biofuel raw materials and is conducive to bringing their production costs to an end-user level acceptable to consumers.

Where the total use of source material is concerned, the invention is not limited only to the total use of the contained carbohydrate. The method also includes stages allowing the lipid components of the extractive fraction in the wood matter to be recovered. When the source material is treated with water, the filtrate separates a lipid component which can be recovered from the aqueous solution by methods familiar to those skilled in the art. As a result of the acid treatment of the source material, the lipids in the extractive fraction yield free fatty acids which are separated from the filtrate in the form of insoluble alkaline earth metal salts, such as Ca⁺⁺ salts, and which, after separation of the precipitate, are transesterified into alcohol esters. Correspondingly, the alkaline extraction of the source material, produces fatty acid salt from the extractive fraction, which salt is water-soluble and is thus mixed in the filtrate. In the invention, said salt is converted into the form of a water-insoluble salt, such as a Ca⁺⁺ salt, and the precipitate is separated from the aqueous phase and is used to manufacture fatty acid alcohol esterids.

The following examples are intended to illustrate the invention, and they are not to be construed as limiting the scope of this invention in any way. Nor is the invention in any way limited to the microbial strains used. The invention can be implemented not only by means of the strains used but also by means of other strains of the same species or genus, or the strains of other microbial genera or species or by means of genetically modified microbe strains. Lipid-producing micro-organisms are commonly available and they can be found in several strain collections, e.g. ATCC, DSM, etc. Lipid-producing microorganisms and lipid production processes using microorganisms (including algae) are described in the literature, for example in the writings: Single Cell Oils, eds. Z. Cohen and C. Ratledge, AOCS Press, 2005 and Microbial Lipids, eds. C. Ratledge and S. G. Wilkinson, vol. 1 and 2, Academic Press, 1988.

### EXAMPLES

### Example 1

100 g each of wood fibre, ground wood, TMP pulp, sawdust and mechanical pulp were kept in 1 litre of boiling water, 90-100° C., for 2 hours. The solutions were filtered away from the fibre material (hereinafter precipitate). The precipitates were recovered and treated using various alternatives to increase the monosaccharide yield according to one of the alternatives presented in Examples 4 or 5. The carbohydrate yields of the solutions ranged from 2% to 6% depending on the source material, being typically 4% when spruce fibre ground at a high temperature (over 170° C.) was used as source material. Some of the solutions were reused without preceding evaporation for extracting the following batches of fibre and some of the solutions were concentrated by evaporation until a dry-matter content of about 20% by weight was achieved. The concentrated filtrates were recovered for the production of single-cell lipid. The dissolved carbohydrates were typical for the tree species extracted, dividing into hexoses and pentoses.

### Example 2

Wood fibre, ground wood, recycled fibre, TMP pulp, sawdust and mechanical pulp were each added to one litre of 5% alkaline solution (NaOH) and stirred at 90-100° C. for 3 hours. Filtrations were carried out at normal temperature and the precipitates were subjected to further treatment for the production of monosaccharides according to Example 4. The extraction solutions were treated so that a part of each solution was reused in treating the next batch of fibre, a part was neutralized, and a part was conducted to hydrolysis according to Example 4 since some lignin as well as some oligo- and polysaccharides had dissolved in the alkaline extraction solutions. The solutions obtained from different source materials by the described alkaline treatment had retained an average of 5% of the weight of the source material.

The precipitates obtained in the original alkaline treatment were also retreated with alkali by repeating the previous alkaline treatment and grinding for 6 minutes at a pressure of 8 bar, at a temperature of 170° C. Using this further grinding, the amount of material dissolving in the alkaline solution could be increased to an average of 27% of the original amounts of source materials. The solutions became black and, following neutralization, hydrolysis and concentration, they were treated with activated charcoal and ion exchanger for decolourization. The mixtures were recovered and used to produce single-cell lipid.

### Example 3

100 g each of wood fibre, ground wood, TMP or MDF pulp, sawdust, mechanical pulp, recycled fibre and neutralized extract prepared according to Example 2 were hydrolyzed in one litre of 5% mineral acid, pH 1, at 90-100° C. for 3 hours. The precipitates were separated from the solutions by filtering. The solutions contained monosaccharides on average 10% of the amounts of source material. Each solution was treated so that a part was neutralized, filtered and concentrated by evaporation until a monosaccharide amount of about 20% by weight was achieved, and the concentrated solutions were recovered for the production of single-cell lipid. A part of the solutions was reused as such for hydrolysis of the following batches of source material as described in this example. The precipitates were further treated to increase the monosaccharide yield according to the following Example 5.

### Example 4

125 g each of wood fibre, ground wood, TMP pulp, MDF pulp, sawdust, mechanical pulp, straw, grain husk and precipitate according to Examples 1-3 were added to a sulphuric acid solution having a concentration of 10%, and the acid was allowed to be absorbed for 2 hours. Excess acid was decanted off and the mixtures were conducted into a wing refiner, the temperature was raised by presteaming for 11 minutes without letting the condensate flow out, after which the temperature was raised 160° C. and the pressure to 8 bar and the grinding was performed using the wing refiner. A mixture grinding time of 11 minutes was selected for the spruce fibre. The precipitates were separated from the solutions after the grinding. Each precipitate was divided so that a part was used for incineration and a part was reground to increase the monosaccharide yield according to the following Example 5. Correspondingly, the solutions were neutralized, filtered and concentrated by evaporation until monosaccharide concentrations of about 20% were achieved. These solutions were recovered for the production of single-cell lipid.

### Example 5

1D. 125 g each of wood fibre, ground wood, TMP pulp, sawdust, mechanical pulp, straw and grain fibre as well as remaining precipitates according to Examples 1-4 were added to a 40% sulphuric acid solution and allowed to absorb at room temperature for 2 hours. Excess acid was decanted off and the precipitates were ground in a wing defibrator so that the temperature of the precipitates was raised by presteaming for 11 minutes without letting the condensate flow out. The temperature was then raised to 170° C. and the pressure to 8 bar. The most suitable grinding time for straw and grain fibre was 11 minutes. The precipitates were separated from the solutions. The proportions of dissolved substances were on average more than 50% of the source material. The solutions were neutralized, filtered and concentrated by evaporation, and recovered for the production of single-cell lipid. The precipitates, i.e. the residual fibres, were partly conducted to incineration and partly reground according to this example to increase the monosaccharide yield.

Concentrated sulphuric acid (72%) was also separately absorbed into the above described source materials, the treatment time being 2 hours, at room temperature. Subsequently, the acid was diluted to 5% and boiled at normal pressure at 100° C. for 4 hours. Further treatment and use of the generated fractions took place as above.

### Example 6

Monosaccharide suitable for producing single-cell lipid can be produced from chaff, straw and grain husk by direct hydrolysis using 5% acid or be impregnated with a stronger one and hydrolyzed in a 5% solution or first be treated with alkali and the hemicellulose and cellulose can be hydrolyzed separately as presented in the above examples. These treatments can be combined with impregnation treatments with acid or alkali and subsequent repeated grindings in a wing or disc refiner to produce thermo-mechanical pulp. In this example, however, chaff, straw and grain husk (125 g each) were pre-steamed for 11 minutes at a pressure of 8 bar and treated into thermo-mechanical pulp in a wing refiner at a temperature of 170° C. The generated thermo-mechanical pulps were hydrolyzed in 5% acid (sulphuric acid) in a volume of one litre at 90-100° C. at normal pressure for 4 hours. The solution fractions were separated by filtration and neutralized. Subsequently, the solutions were concentrated and recovered for the production of single-cell lipid. The monosaccharide yield was on average 50% of the source material. To increase the carbohydrate yield, the precipitates remaining after the filtration were thermo-mechanically re-treated according to Example 5 and a part was conducted to incineration.

### Example 7

Chaff, straw and grain husk (16 kg each) were each treated in 100 litres of alkaline solution (1.2 M NaOH) at 90-10° C. for 4 hours. The mixture was divided into a precipitate and a solution. On average 49-57% of the dry matter of the fed source materials ended up in the solutions. The alkaline solutions were rendered 5% in terms of the acid, using sulphuric acid, and they were hydrolyzed as in Example 6. Mixtures of xylose and arabinose, which also contained small amounts of glucose and galactose, were mainly formed. The monosaccharide content of the solutions became 23-33% of the fed source material. The colour of the solutions was reduced by treatment with activated charcoal before they were recovered for the production of single-cell lipid.

The precipitates obtained in the filtration were impregnated in 5% acid (sulphuric acid) and ground in a wing refiner for 11 minutes at a pressure of 6 bar and a temperature of 150° C. The precipitates degraded into soluble carbohydrates, mainly glucose and galactose, which after neutralization and filtration were recovered for the production of single-cell lipid. Parts of the precipitates remaining after the filtrations, carried out after the acid hydrolysis, were still re-ground and then hydrolyzed in 10% acid according to Example 4. Solutions were obtained after these treatments, which contained monosaccharides in a total of 55-65% of the source material. The solutions were recovered for the production of single-cell lipid.

### Example 8

Steam-dried beet pulp was poured into a tub and enough boiling water was poured on top of it so that the water covered the beet pulp. The solution was allowed to cool before separating the water from the solids by decanting. 3.6 mg/ml of dry matter had dissolved in the water. The aqueous fraction was partly used to treat a new batch and partly, after concentrating, recovered for the production of single-cell lipid. The obtained solid fraction, the precipitate, containing 16.7% of dry matter, was weighed 748 g, corresponding, as dry matter, to 125 g of dried beet pulp, was impregnated with 0.4M phosphoric acid (H₃PO₄, 500 ml) at room temperature for 18 hours. Excess acid solution was decanted off and pre-steamed for 11 minutes before initiating the thermo-mechanical grinding. The mixture was ground for 11 minutes using a wing refiner. During grinding, the pressure was 8 bar and the temperature was initially 172° C. and, when 10 minutes of grinding time had lapsed, 162° C. The mixture was removed from the refiner and filtered. The solution fraction, 2.26 litres, contained 2.75% dry matter. 50% of the dry matter fed to the grinding was present in the solution after the filtration. The solution contained mainly monosaccharides, glucose, galactose, arabinose and xylose. In addition, minor amounts of oligosaccharides in the molecular weight range of 500 to 3000 and higher molecular weight compounds were observed. The solution fraction was neutralized, concentrated and recovered for the production of single-cell lipid.

The solid fibre pulp, the precipitate (125 g dry matter), obtained from the above treatment of the beet pulp was impregnated in 15% hydrochloric acid at room temperature for 4 hours. Excess acid solution was decanted off and the pulp was reground under the same conditions. After the filtration stage, it was observed that an additional 28% of monosaccharides had entered the solution phase. The precipitate was discarded.

### Example 9

The source material was thermo-mechanically treated spruce fibre, of which 46.8 g (about 1 litre in volume) was weighed, and 0.2N NaOH was added on top of it, to which a further 5.6 g of Na₂CO₃ and 1.3 g of MgSO₄ 6H₂O (500 ml) was added. The mixture was heated to 50° C., yielding minor amounts of glucose, xylose, galactose, arabinose, mannose and additionally oligomers in the solution. The pH of the solution was about 11, it was filtered and washed with water. The washing and the filtrate were combined and the solution was evaporated to 320 ml. The amount of dissolved substance was 4.5% (dry matter) of the source material. To increase the amount of monosaccharides, acid was added to the solution, resulting in a precipitate. The precipitate was separated from the solution, the latter being recovered and used to produce single-cell lipid. The precipitate was hydrolyzed into monosaccharides in 5% acid as described in Example 3. Using the hydrolysis, 1% monosaccharides was generated from the mass of the precipitate, which monosaccharides entered the solution and were, after decolourization, recovered for the production of single-cell lipid.

### Example 10

A 45 g batch (about 1 litre) of thermo-mechanically treated spruce fibre was weighed, on top of which was added 0.2 N NaOH, to which was added a further 5.6 g of Na₂CO₃ and 1.3 g of MgSO₄ 6H₂O (500 ml). The mixture was heated to 50° C., filtered and the precipitate washed with water. The washing and filtrate solutions were combined. To increase the amount of monosaccharides, acid was added to the combined filtrate and hydrolysis was performed according to Example 3. After neutralization, the generated hydrolysis product was recovered for the production of single-cell lipid. The washed precipitate was compressed to 300 ml and 1 litre of citrate buffer, pH 4.8, was added, and cellulase enzyme was added. Oligomers and glucose were generated in the mixture. The enzyme-treated mixture was recovered both as such and as a separated filtrate, a solution, and used to produce single-cell lipid.

### Example 11

A 125 g batch of oat chaff was provided and it was thermo-mechanically ground at 170° C. and a pressure of 8 bar for 2 min. After the treatment, the precipitate was separated from the solution by filtration. The solution was recovered for use in producing single-cell lipid. A 7 g batch of precipitate (fibre) was used and rendered 19% in strength in terms of sulphuric acid and refluxed for 4 hours. Analysis showed that the treatment resulted in the monosaccharides entering the solution in 56% of the source material, mostly as xylose, mannose, glucose galactose and arabinose. The solution was recovered for use in producing single-cell lipid.

### Example 12

400 g of oat chaff was weighed and 3 litres of water and 200 g of NaOH were added. The mixture was kept at a temperature of 90-96 C under stirring for 2 hours. The mixture was filtered through a cloth and the fibres were separated. The filtrate fraction (50 ml) was neutralized and its pH adjusted to 4.8 with a citrate buffer (40 ml), multifect xylanase (10 ml) is added and the mixture was thermostated to 50° C. Sugars were released into the solution, of which 25.2% were xylose and 11.8% were arabinose. There were also oligomers in the solution after a reaction time of 50 hours. The entire mixture was recovered for use in producing single-cell lipid.

### Example 13

Beet pulp, 125 g, was impregnated with 0.4 M sulphuric acid and excess acid was decanted off after 12 hours. The pulp was transferred into a wing refiner, in which it was pre-steamed for 4 min and ground at 150° C. at a pressure of 6 bar for 11 minutes. The mixture was neutralised and citric acid was added until the pH dropped to 4. 10 ml of pectinase 4450 U, i.e. 178 mg/ml of protein (Sigma), were added and the reaction was allowed to proceed for 24 hours at 25° C. After the reaction, a part of the mixture was recovered for the production of single-cell lipid and a part was filtered. The precipitate obtained from the filtration was returned to the grinding stage and the solution fraction resulting from this treatment was treated with activated charcoal, 2 g/litre, and then conducted to an anion exchange column. The monosaccharide solution obtainable from the column was evaporated to a concentration of 20% in terms of dry matter, was recovered for the production of single-cell lipid.

### Example 14

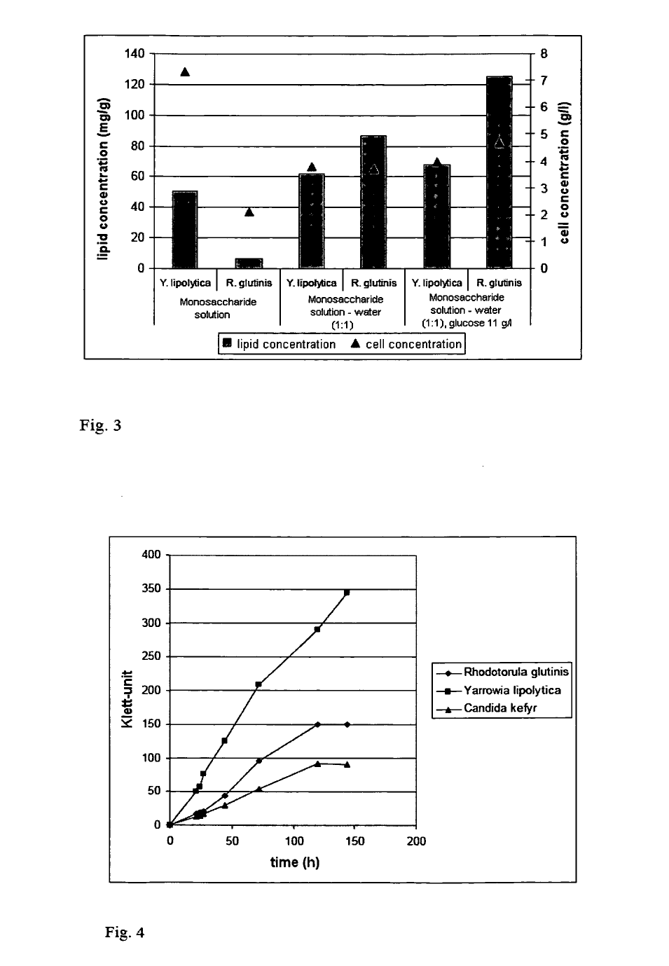

Oat chaff was treated according to Example 7, yielding a mixture containing glucose at 23.8 g/L, xylose at 93.3 g/L, arabinose at 37.1 g/L and galactose at 9.0 g/L. This mixture was added as a culture medium for the lipid-synthesizing yeasts Yarrowia lipolytica ATCC 20373 and Rhodotorula glutinis TKK 3031 as such, as a 1:1 dilution and as a corresponding dilution supplemented with glucose at 11 g/L. The culture period was 68 hours, the temperature 28° C., shaking at 250 rpm and culture volume 50 ml. As is observed in FIG. 3, the yeasts were able to grow and synthesize lipid without requiring the addition of other nutrients.

### Example 15

Three yeasts, Rhodotorula glutinis, Yarrowia lipolytica and Kluyveromyces marxianus (Anam. Candida kefyr) ATCC 42265, were cultured solely with xylose as the carbon source. The culture medium contained xylose at 20 g/L, yeast extract at 10 g/L and peptone at 20 g/L. The culturing was performed in a volume of 50 ml and at 25° C. under shaking at 200 rpm. FIG. 4 shows that all three strains are able to utilize pentose sugar as a carbon source.

1. A method of producing a lipid or a lipid mixture from an organic source material comprising a polysaccharide, which is selected from a group comprising cellulose, hemicellulose, starch, all of these, any mixture thereof or the degradation products thereof or non-starch polysaccharide, characterized by comprising:
a) treating the source material with a substance, which is selected from a group comprising:
i) water,
ii) acid, and
iii) alkali,
after which the precipitate and the filtrate are separated, and the precipitate obtained from said treatments is subjected to mechanical or thermo-mechanical grinding as such or in the presence of water, acid or alkali, and the precipitate and the filtrate are separated and, alternatively, subjecting the precipitate one or more times again to the treatment(s) and/or grinding of any of sections i), ii) or iii), and
b) contacting a lipid-producing microorganism with the filtrate thus obtained or with several obtained filtrates or with the precipitate, or with any combination obtained from these and, optionally, with the source material, in a culture medium, whereby the microorganism cells begin to produce lipid, and
c) recovering the lipids.

2. The method according to claim 1, characterized by treating the precipitate optionally also using a method comprising one or more of the following steps:
d) treating the precipitate obtained from section a) with a strong acid and separating the precipitate and the filtrate or, alternatively,
e) acidifying the precipitate obtained from section a) or d) and grinding it mechanically or thermo-mechanically, and separating the precipitate and the filtrate or, optionally, treating the precipitate obtained from any of steps a), d) or e) again one or more times using the method according to any of steps a), d) or e) in an optional order,
f) contacting the lipid-producing microorganism with the filtrate or the precipitate obtained from section a), d) or e) or with any combination obtained from these and, optionally, with the source material, in the culture medium, whereby the microorganism cells begin to produce lipid, and
g) recovering the lipids.

3. The method according to claim 1, characterized in that the source material originates from the mechanical or thermo-mechanical treatment of wood or from a cultivated plant.

4. The method according to claim 3, characterized in that the source material is treated with water or acid.

5. The method according to claim 1, characterized in that the source material originates from a source selected from a group comprising recycled fibre, sugar beet pulp, chaff, straw, bran, grain granules, whole cultivable crop, cultivated plant, TMP pulp and MDF pulp.

6. The method according to claim 5, characterized in that the source material is treated with acid.

7. The method according to claim 1, characterized in that the source material originates from a source selected from a group comprising sawdust, refined mechanical pulp, chaff, straw, TMP pulp, MDF pulp, sugar beet pulp and cultivated plant containing no essential amount of starch.

8. The method according to claim 7, characterized in that the source material is treated with alkali.

9. The method according to claim 1, characterized in that the source material originates from a source selected from a group comprising microbial mass, swampy or submerged biomass, biomass from the catchment areas of a cellulose mill, biomass from municipal waste and biomass from municipal sewage.

10. The method according to claim 1, characterized in that the filtrate contains at least 0.5-1% by weight, up to 20-30% by weight, preferably 4-5% by weight, of sugars usable in lipid production.

11. The method according to claim 1, characterized in that the treatment of any step a) is carried out one or more times.

12. The method according to claim 2, characterized in that the treatment of any of steps d) or e) is carried out one or more times.

13. The method according to claim 11, characterized in that, when treating the source material with alkali in section a) iii), the precipitate obtained is re-treated with acid, and the precipitate and the filtrate are separated.

14. The method according to claim 2, characterized in that, when treating the fibre-containing precipitate with a strong acid, the precipitate obtained is ground again mechanically or thermo-mechanically.

15. The method according to claim 1, characterized in that one or more enzymes are added to the biomaterial treatment solution.

16. The method according to claim 1, characterized in that the filtrate obtained from any of the process steps is treated further using methods, which render the filtrate more suitable for the growing of microorganisms, such as decolourization methods, adjusting the pH and/or removing or adding water.

17. The method according to claim 1, characterized in that the source material is pre-treated mechanically, thermo-mechanically, physically, chemically, biologically or by combinations of these treatments.

18. Use of the lipid or the lipid mixture produced using the method according to claim 1 as the raw material of manufacturing biofuel.

19. Use of the lipid or the lipid mixture produced using the method according to claim 2 as the raw material of manufacturing biofuel.

20. A biofuel, characterized in that lipid is used for its manufacture, which lipid is produced using the method according to claim 1.

21. A biofuel, characterized in that lipid is used for its manufacture, which lipid is produced using the method according to claim 2.

22. A method for purifying municipal sewage, characterized in that the municipal sewage is treated using the method according to claim 1.

23. A method for purifying municipal sewage, characterized in that the municipal sewage is treated using the method according to claim 2.
